(19) United States
(12) Patent Application Publication
(10) Pub. No.: US 2005/0201962 A1
(43) Pub. Date: Sep. 15, 2005

(54) Modified Porphyran

(75) Inventors: Hiroshi Hagino, Tokyo (JP); Teruyoshi Kubota, Tokyo (JP); Masanobu Saitoh, Chiba-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SHIRAKO CO., LTD., Tokyo (JP)

(21) Appl. No.: 11/078,617

(22) Filed: Mar. 14, 2005

(30) Foreign Application Priority Data
Mar. 15, 2004 (JP) 2004-72426

(51) Int. Cl. C11D 7/42; A61K 7/50

(52) U.S. Cl. 424/70.1; 510/130

(57) ABSTRACT

Porphyran with modified bioactivity and physical property, method of making the modified Porphyran and use of modified Porphyran. Porphyran with modified bioactivity and physical property is produced by dissociating a salt from a sulphate group in Poryphran and removing it, making Porphyran having a free sulphate group wherein a salt is dissociated. Further, Porphyran with modified bioactivity and physical property is produced by dissociating a salt from sulphate group in Porphyran and removing it, and then altering the sulphate group, with the use of a given salt, into a sulphate salt of the given salt. The modified Porphyran also can be obtained by converting a salt of a sulphate group in Porphyran into a sulphate salt of a given salt by ion exchange. The modified Porphyran be added to cosmetics, food, and drink, and used as cosmetic, food and drink having inhibitory activity against hyaluronidase activity.

### TECHNICAL FIELD

The present invention relates to a Porphyran with modified bioactivity and physical property, a method for production thereof, and a use thereof.

### BACKGROUND ART

Porphyran is a polymer of acidic saccharide containing sulphate groups extracted from algae bodies of Rhodophyta Porphyra; e.g., Porphyra yezoensis, Porphyra tenera, and Porphyra suborbiculata which traditionally have been used in Japan as food such as dry sheet laver or stewed laver in soy sauce. The Porphyran contains D-galactose, 3, 6-anhydro-L-galactose, and 6-O-methyl-D-galactose, and is similar to agar in structure, though it does not show gelation ability as agar does because it has much galactose-6-sulphuric acid. Further detailed analysis of its structure has been performed by instrumental analysis or the like using NMR and so on, leading to the report that Porphyran comprises agarobiose of agarose as a base unit, where 2-2.5 units, one ends of which are sulphated, are proximate partially, and it contains approximately 6-11% of sulphate groups.

Meanwhile, Porphyran has been reported that it has following bioactivities similar to those of other sulphated polysaccharide: metallic adsorption ability (Ishibashi, Changes in the physical properties of Asakusanori Porphyran during heated process, Kyushu Women's University bulletin, 1990, 26, 15-21), improvement of intestinal flora (Kawazu et al., Effect of polysaccharides of “Susabinori” Porphyra yezoensis on intestinal flora, Nippon Suisan Gakkaishi 1995, 61, 56-69), Antitumor Activity (Noda et al., Studies on the antitumor activity of marine algae, Nippon Suisan Gakkaishi, 1989, 55, 1259-1264, and Nodaet al. , Antitumor activity of polysaccharides and lipids from marine algae, Nippon Suisan Gakkaishi, 1989, 55, 1265-1271), and immunostimulatory activity (Yoshizawa et al., Activation of marine macrophages by polysaccharide fractions from marine algae (Porohyrayezoensis), B. B. B., 1993, 57, 1862 -1866, Yoshizawa et al., Study on polysaccharides having immunostimulatory activity derived from Porphyra, Nippon Shokuhin Kogyo Gakkaishi, 1994, 37, 25-30, Yoshizawa et al., Macrophage stimulation activity of the polysaccharide fraction from a marine Algae (Porphyra yezoensis): Structure-function relationships and improved solubility, B. B. B., 1995, 59, 1933-1937).

Potential utility value of Porphyran as an effective bioactive substance is so expected that its use in food, drink, cosmetic and the like is devised. Further, as to its physical properties, it is known that Porphyran does not show gelation ability or significant viscosity, but its gelation ability is induced when desulphated by alkaline treatment. In addition, use of Porphyran as emulsifying agent for oil and emulsion stabilizer is also devised by utilizing its amphipathic property.

On the other hand, some methods for producing or purifying Porphyran have been disclosed. These are examples of such disclosed methods: a method for producing Porphyran by precipitating Porphyran from hydrothermal extract solution from Porphyra by alkaline metal salt or alkaline earth metal salt, and water-soluble organic solvent (Japanese Patent Publication No. 8-841), a method for producing Porphyran by precipitating hydrothermal extract solution from Porphyra with alcohol and treating it with size exclusion chromatography (Japanese Laid-Open Patent Application No.11-310603), a method for purifying Porphyran by alkalizing the hydrothermal extract solution from Porphyra with sodium hydrate, adding precipitant (invert soap: alkyl trimethyl ammonium compound), and washing with ethanol (Japanese Laid-Open Patent Application No. 10-60002), and a method for producing Porphyran by conducting hot water extraction, treating the resulting marine algae hydrothermal extract solution with acetic acid and/or acetate salt, adding ethanol to precipitate Porphyran, and washing it (Japanese Laid-Open Patent Application No. 10-60003).

In relation to a method for producing or purifying Porphyran, another method is disclosed, in which hydroscopic ethyl alcohol is added to dried algae body of Porphyra, and the resultant is subjected to wash treatment, water is added to the residual solid content, and the resultant is heated until boiled, then filtered, the filtrate is concentrated, the concentrated solution is added alkaline metal salt or alkaline earth metal salt of hydrochloric acid or lactic acid, then fractional precipitation is conducted with ethyl alcohol, and the precipitate of fraction at alcohol concentration of 50% (v/v) is retrieved (Japanese Laid-Open Patent Application No. 2004-27192).

Cited Document in the Above Description of Background Art

(Patent Document 1) Japanese Patent Publication No. 8-841.

(Patent Document 2) Japanese Laid-Open Patent Application No. 10-60002.

(Patent Document 3) Japanese Laid-Open Patent Application No. 10-60003.

(Patent Document 4) Japanese Laid-Open Patent Application No. 11-310603.

(Patent Document 5) Japanese Laid-Open Patent Application No. 2004-27192.

(Non-patent Document 1) Ishibashi, Changes in the physical properties of Asakusanori Porphyran during heated process, Kyushu Women's University bulletin, 1990, 26, 15-21.

(Non-patent Document 2) Kawazu et al., Effect of polysaccharides of “Susabinori” Porphyra yezoensis on intestinal flora, Nippon Suisan Gakkaishi 1995, 61, 56-69.

(Non-patent Document 3) Noda et al., Studies on the antitumor activity of marine algae, Nippon Suisan Gakkaishi, 1989, 55, 1259-1264.

(Non-patent Document 4) Noda et al., Antitumor activity of polysaccharides and lipids from marine algae, Nippon Suisan Gakkaishi, 1989, 55, 1265-1271.

(Non-patent Document 5) Yoshizawa et al., Activation of marine macrophages by polysaccharide fractions from marine algae (Porohyra yezoensis), B. B. B., 1993, 57, 1862-1866

(Non-patent Document 6) Yoshizawa et al., Study on polysaccharides having immunostimulatory activity derived from Porphyra, Nippon Shokuhin Kogyo Gakkaishi, 1994, 37, 25-30.

(Non-patent Document 7) Yoshizawa et al., Macrophage stimulation activity of the polysaccharide fraction from marine Algae (Porphyra yezoensis): Structure-function relationships and improved solubility, B. B. B., 1995, 59, 1933-1937.

### DISCLOSURE OF THE INVENTION

Porphyran is not commercially available currently; though it was once tried to be extracted from Porphyra marine algae and used and it was also sold in 1980's. Although high cost of laver which is raw material of Porphyran extraction was cited as a reason of commercial unavailability, Porphyran is abundantly contained in laver with inferior quality, and raw laver, dried laver, roasted laver, and even laver being unsuitable for food due to its discoloration can be used as raw material of Porphyran, therefore the difficulty with material cost has been solved. As to the reason why Porphyran has not yet been applied in practical use though the difficulties with material cost has been already solved, followings are suggested: explosion-proof facilities are required in known methods for producing Porphyran because an organic solvent is used in the process of purification, it brings the cost increase for purification, and its clear usage worth the cost has not yet been established.

Hence, the task of the present invention is to provide modified Porphyran by producing Porphyran with modified bioactivity and physical property by a convenient method, in addition, to provide a use practically useful by applying the modified Porphyran.

A keen study to solve the above-mentioned task by the present inventors found that Porphyran with modified bioactivity and physical property can be produced by dissociating a salt from a sulphate group in Porphyran, removing it, thereby making Porphyran having a free sulphate group wherein a salt thereof is dissociated. Further the present inventors found that Porphyran with modified bioactivity and physical property can be produced, by dissociating a salt from a sulphate group in Porphyran and removing it, and then altering the sulphate group, with the use of a given salt, into a sulphate salt of the given salt; or by converting a salt of a sulphate group in Porphyran into a sulphate salt of a given salt by ion exchange. The present invention has been thus completed. In the present invention, as for dissociating the salt of the sulphate group in Porphyran and removing it, or performing ion exchange of the salt of the sulphate group in Porphyran, a cation-exchange resin can be used. As for sulphate salt of the given salt converted in the present invention, sodium salt, potassium salt, calcium salt, arginine salt, ornithine salt, histidine salt and so on of sulphuric acid can be exemplified.

When described in detail, the present inventors found that Porphyran having various properties can be obtained, by producing it via a process of exchanging the ion of the sulphate group using crude Porphyran solution obtained by extraction from Porphyra marine algae. The present inventors also found that the bioactivities and physical properties of Porphyran change significantly by exchanging the ion of the sulphate group; the bioactivities and physical properties of Porphyran depend largely on the type of salt of its sulphate group. Inhibitory activity against hyaluronidase was expressed as a novel property of Porphyran by setting the sulphate group as a given one, leading to that the practically useful application of Porphyran as cosmetic material.

Porphyran has been retrieved conventionally as precipitate by a hydrophilic organic solvent and the like after extraction in production of Porphyran from laver and its purification. In this case, Porphyran in the hydrothermal extract tends not to precipitate, unless an organic solvent such as ethanol at least in equal proportions to the extraction, is added to the hydrothermal extract from Porphyra. A result obtained from the experiment conducted by the present inventors also showed that comparably low-molecular weight Porphyran tends not to precipitate, thus a precipitation/retrieval method has another difficulty in yield rate. Further, complicated processes had to be taken for precipitation of Porphyran, e.g., a condition that precipitate was left standing for a long time at low temperature was required. Additionally, Porphyran obtained by the conventional method still included many problems in its utilization from the point of practical use, because it had less characteristic bioactivities than other dietary fibers as well as it had a defect in stability for its use as solution.

Porphyran is stable when its sulphate group is salt. Sodium, potassium or other salts exist in mixture in the extract from Porhyra marine algae. The present inventors discovered that the physical properties and bioactivities of the salt depend largely on their types, while in the past, there was neither example in which it was found that physical properties and bioactivities of Porphyran depend on types of salts binding to sulphate groups, nor example in which physical properties or bioactivities of Porphyran were modified by exchanging its salt, in the past. It is because salt was used from the viewpoint of how to precipitate Porphyran in process of production in any of the known examples.

Therefore, the present inventors provide Porphyran showing various kinds of nature, by treating hydrothermal extract from Porphyra marine algae containing Porphyran by cation-ionic exchange, freeing the salt from the sulphate group of Porphyran, and then making the salt as an optional salt. That is, free Porphyran is led by treating crude Porphyran solution with a cation-exchange resin and the like, salt exchanges is further performed to exchange the resultant with various desired salts, and thus intended Porphyran can be obtained. By taking a method for treating the crude Porphyran solution with a cation-exchange resin and the like, Porphyran with modified bioactivity and physical property, can be produced by a simple method.

On the contrary, as to the conventional use of Porphyran in cosmetic field, its viscosity and effect of retaining moisture were focused so that the use was aimed to improve skin condition after applying it to skin: providing smooth or moisturized skin, or removing overproduced oil. Recently, knowledge referring to physiological activities of Porphyran as cosmetics has been found, whereas little has been elucidated as to its specific physiological activity and biochemical activity on skin.

Inhibitory activity against hyaluronidase was newly found as characteristic of Porphyran by the present invention and it was confirmed that the inhibitory activity against hyaluronidase contained in Porphran is enhanced by the modification of the present invention. Hyaluronic acid is one of matrix components widely existing in connective tissue of mammals; not only physical activities such as retention of cells, water-retention of skin, and lubrication of arthrosis, but also biochemical activities such as control of function of cells including vascular endothelial cells, polymorphonuclear leukocytes, macrophages and the like have been taken notice recently. Hyaluronic acid in vivo is observed its metabolic turnover in relatively short term. The balance between activities of synthetase and catabolic enzyme of hyaluronic acid is maintained under normal condition. However the loss of this balance along with aging and increase of hyaluronidase activity that is a catabolic enzyme of hyaluronic synthesis causes the loss of softness and lubricity of tissue and age change such as wrinkles on skin. Because increase of hyaluronidase activity is confirmed in skin senescence, allergic dermatitis, rheumatic arthritis, and tumor growth, hyaluronidase-inhibitory substance is expected to suppress changes and diseases along with such senescence.

Further as involvement of histamine in onset of allergy is known and possibility of involvement of hyaluronidase in freeing histamine from mast cells was reported, inhibitory activity against hyaluronidase is regarded as an indicator of antiallergy activity. Development of hyaluronidase-inhibitory substance having high safety for application to human body such as to skin and the like as well as having excellent inhibitory activity against hyaluronidase was expected. Inhibitory activity against hyaluronidase was newly discovered in Porphyran in the present invention. Inhibitory activity against hyaluronidase could be found in Porhyran as well obtained from the conventional method for producing, however, remarkable bioactivity was confirmed in Porphyran produced by the present invention.

Meanwhile, the application of Porphyran whose bioactivities such as cholesterol-lowering activity or immunostimulatory activity is utilized is devised in food industrial field. However, experiments conducted by the present inventors revealed that it is the most important to ensure the stability of its physical properties in Porphyran-added food. That is, Porphyran prepared by the conventional method for producing was instable in physical properties such as solubility, viscosity in solution, and stability, so that it comprised various defects in actual processing. In the present invention, it was revealed that those physical properties of Porphyran depend on the salt of the sulphate group. Further, present invention made it possible to provide a convenient method for producing Porphyran suitable for various processing by salt exchange response.

As aforementioned, the present inventors could produce Porpyran with modified bioactivity and physical property by a simple method; could provide the practically useful application of Porphyran by using the modified Porphyran. Cosmetics, food or drink added the Porphyran show preferable physical properties in quality of products, and they also provide Porphyran cosmetic, food, or drink where novel bioactivities with enhanced effects of Porphyran by the modification of Porphyran of the present invention are expressed apparently.

Specifically, the present invention relates to a method for producing modified Porphyran, wherein the method includes a process of dissociating a salt from a sulphate group in Porphyran and removing it (“1”); a method for producing modified Porphyran, wherein the method includes a process of producing Porphyran by dissociating a salt from a sulphate group in Porphyran and removing it, and then altering the sulphate group, with the use of a given salt, into a sulphate salt of the given salt; or by converting a salt of a sulphate group in Porphyran into a sulphate salt of a given salt by ion exchange (“2”); the method for producing modified Porphyran according to “1” or “2”, wherein salt dissociation from the sulphate group in Porphyran and removal of the salt, or conversion of the salt of sulphate group in Porphyran by ionic exchange is performed with a cation-exchange resin (“3”); and-the method for producing modified Porphyran according to “2” or “3”, wherein the sulphate salt of the given salt is sodium salt, potassium salt, calcium salt, arginine salt, ornithine salt, or histidine salt of sulphuric acid (“4”).

The present invention further relates to a modified Porphyran wherein the Porphyran is produced by the method for producing according to “1”, thereby making the Porphyran having a free sulphate group wherein a salt thereof is dissociated (“5”); a modified Porphyran, wherein it is produced by the method for producing according to any one of “2” to “4”, thereby the salt of the sulphate group in Porphyran is converted to the given salt (“6”); the modified Porphyran according to “6”, wherein the given salt is sodium salt, and shows high stability and enhanced adsorption to skin when it is brought into solution (“7”); the modified Porphyran according to “6”, wherein the given salt is arginine salt, and shows high stability and enhanced adsorption to skin when it is brought into solution (“8”); and a hyaluronidase activity-inhibitory agent wherein its active ingredient is the modified Porphyran according to any one of “5” to “8” (“9”)

The present invention still further relates to a cholesterol-lowering agent wherein its active ingredient is the modified Porphyran according to any one of “5” to “8” (“10”); an immunostimulant wherein its active ingredient is the modified Porphyran according to any one of “5” to “8” (“11”); a cosmetic or a cleanser for hair or body, to which the modified Porphyran according to any one of “5” to “8” is added (“12”); and a food or a drink to which the modified Porphyran according to any one of “5” to “8” is added (“13”).

### BEST MODE OF CARRYING OUT THE INVENTION

The present invention comprises producing Porphyran with modified bioactivity and physical property by dissociating salt from the sulphate group in Porphyran, removing it, thereby making Porphyran having a free sulphate group wherein a salt thereof is dissociated. The present invention further comprises producing Porphyran with modified bioactivity and physical property by dissociating a salt from a sulphate group in Porphyran and removing it, and then altering the sulphate group, with the use of a given salt, into a sulphate salt of the given salt; or by converting a salt of a sulphate group in Porphyran into a sulphate salt of a given salt by ion exchange.

Material for extracting Porphyran used in the method for producing modified Porphyran of the present invention is not especially limited, e.g., any of Porphyra marine algae in addition to Porphyra yezoensis and Porphyra tenera, which are the major cultured varieties of marine algae belong to Rhodophyta Porphyra in Japan, can be used. Meanwhile, they may take any form such as raw laver, dried laver, roasted laver, or the like.

First, extract from Porphyra marine algae is obtained by aqueous medium for producing Porphyran. Temperature and time for extraction from Porphyra marine algae may be selected from the range of 0-200° C. and 1-1440 min, respectively, depending on purpose, but the extraction is preferably performed with selecting from the range of the temperature of 50-150° C. and the time of 5-720 min usually. Additionally, it is advantageous to wash the extract with 60-100% alcohol or acetone or immerse it with aqueous solution containing formaldehyde, acetaldehyde, gultaraldehyde, ammonium or the like before the process of extraction, because it largely reduces contamination to Porphyran by substance having staining properties or protein.

Next, treatment of exchanging salts of the sulphate group of Porphyran which is an essential process of the present invention will be described. Since Porphyran in hydrothermal extract from the algae belong to Porphyra exists in the form of various salts such as sodium, potassium, or the like, treatment with a strongly acidic cation-exchange resin is preferable as the process of dissociating salts from most of the sulphate groups and removing the salts. Further electrodialysis, treatment with an anion exchange-resin, or the like can also be used at the same time. As a method for exchanging salt, a strongly acidic cation-exchange resin adjusted to the form of base pair can be used. Alternatively, it is possible to yield exchange response by neutralization or the like with basic substance after treated with a strongly acidic ion-exchange resin adjusted to H+ type.

The nature of Porphyran depends largely on base used; therefore an appropriate base should be selected in order to obtain Porphyran having desired nature. As for a sulphate salt used in the present invention, sodium salt, potassium salt, calcium salt, arginine salt, ornithine salt, histidine salt, and the like of sulphuric acid can be exemplified. When the salt used in the sulphate group of Porphyran is sodium salt or arginine salt, Porphyran, which shows high stability and transparency, excellent moisturizing ability, as well as high adsotbability to skin, can be obtained, when it is used in the form of solution. Accordingly it is possible to apply it to aqueous cosmetics, drink, and so on.

On the other hand, when the used salt of the sulphate group of Porphyran is histidine salt or ornithine salt, Porphyran, which shows low adsorbability to skin, hair, or the like and gives refreshment organoleptically, can be obtained and thereby the Porphyran can be applied to hair care and body care product. When the salt of the sulphate group of Porphyran is alkaline metal salt such as potassium salt or lithium salt, or alkaline earth salt such as calcium salt or magnesium salt, Porphyran has low stability in the form of solution, and may cause aggregation and precipitation or the like, hence it should be avoided to be applied to liquid product such as drink. Meanwhile, the present invention revealed that inhibitory activity against hyaluronidase is exponentially enhanced by exchange response of the salt of the sulphate group in any Porphyran.

Purified Porphyran per se, whose salt of the sulphate salt is converted by ion-exchange response in the present invention, can also be used through the addition to cosmetics, food, drink, or the like. Further the processes such as molecular-mass fragment, decolorization/deodorization treatment, desalting treatment, condensation, drying and the like which are applied as methods for producing/purifying polysaccharide, may be employed according to the necessity when appropriate. The modified Porphyran of the present invention can be used as hualuronidase activity-inhibitory agent, cholesterol-lowering agent, or immunostimulant by formulating Porphyran to take the form of solid or liquid agent or the like. The modified Porphyran of the present invention can be formulated and used as food with health-promoting benefit.

The present invention will be described more specifically with examples below, but the technical scope of the present invention will not be limited to these exemplifications.

### EXAMPLE 1

Preparation of Porphyran Solution

Dried marine algae of Porphyra yezoensis from the sea of Ariake, Kyushu (Fukuoka, Nagasaki, Kumamoto, and Saga prefectures) was used as Porphyran material. Extraction was performed at 95° C. for 3 h with 1 kg of the dried marine algae in 12 kg of boiling purified water. Then, after extraction residue was filtered and separated through a 50 mesh shifter, diatom earth and activated charcoal equivalent to 1% and 5% of the extract respectively were added to the extract, centrifugation was conducted (14,000 rpm, 10 min), extraction residue, substances having staining properties, and odor were completely removed, and then each process of purification shown in Table 1 was performed. Respective resulting Porphyran solutions from respective processes were concentrated so that the concentrations of solid content (Bx.) went up to 3%, and dispensed to containers having screw lids by 50 ml. After autoclaved (120° C., 20 min), they were subjected to conservation test for one month under the environment at 40° C. As a result, occurrence of precipitation was observed during conservation period in process examples 1 and 2. Deterioration in viscosity was observed in process example 3, and slight sedimentation was observed in process examples 5and 6, while neither precipitation nor sedimentation was observed in other process examples such as 4, 7, 8, and 9.

TABLE 1

| | Process |
|---|---|
| Process Example 1 | Untreated |
| Process Example 2 | Precipitating Porphyran by adding |
| | ethanol in advance, and redissolving |
| | it with distilled water |
| Process Example 3 | No neutralizing, |
| | Cation-exchange process only |
| Process Example 4 | Neutralizing with NaOH after |
| | cation-exchange process |
| Process Example 5 | Neutralizing with KOH after |
| | cation-exchange process |
| Process Example 6 | Neutralizing with Ca(OH)₂ after |
| | cation-exchange process |
| Process Example 7 | Neutralizing with arginine after |
| | cation-exchange process |
| Process Example 8 | Neutralizing with ornithin after |
| | cation-exchange process |
| Process Example 9 | Neutralizing with histidine after |
| | cation-exchange process |

Test Example 1) Determiniation of Inhibitory Activity Against Hyaluronidase.

The Porphyran solutions prepared in Example 1 were prepared so as to have concentrations of 1.024, 0.256, 0.064, 0.016, 0.004, and 0.001 mg/ml respectively by conversion with solid content (Bx.) and used as test solutions. Then, 0.2 mL of the test solution was added to 0.1 mL of hyaluronidase solution (330 units/mL) and the resulting solution was incubated for 20 min at 37° C. Further, 0.2 mL of enzyme activating solution (2.5 mM of calcium chloride/2H₂O) was added and the resulting solution was incubated for 20 min at 37° C., and then 0.5 ml of 0.08% hyaluronic acid solution was added, followed by continuous incubation for 40 min. Enzyme activity was determined by Morgan-Elson method.

The reaction was stopped by adding 0.1 mL of 0.4 N of NaOH to reaction samples, and then the reaction samples were added 0.1 mL of potassium borate solution and heated for 3 min in boiling water bath. After cooled with running water, they were added 3 mL of p-dimethylaminobenzaldehyde solution and well agitated, reacted for 20 min at 37° C., and determined their absorbency (OD) at 585 nm. The inhibitory rate against hyaluronidase activity was calculated by using following formula;
inhibitory rate against hyaluronidase activity (%)=A-C/A-B,
wherein:
OD [A] is absorbency of solution in similar operation using purified water instead of the test solution,
OD [B] is absorbency of solution in similar operation using buffer instead of enzyme solution, and

OD [C] is absorbency of solution in similar operation using test solution. The result was shown in Table 2. As shown in Table 2, inhibitory activity against hyaluronidase was confirmed in Porphyran, among which particularly strong inhibitory activity was observed in process examples 4-9 which belong to the present invention.

TABLE 2

| | Inhibitory Activity |
|---|---|
| | against Hyaluronidase |
| | IC50 (mg/ml) |
| Process Example 1 | 0.520 |
| Process Example 2 | 0.610 |
| Process Example 3 | 0.200 |
| Process Example 4 | 0.030 |
| Process Example 5 | 0.020 |
| Process Example 6 | 0.010 |
| Process Example 7 | 0.040 |
| Process Example 8 | 0.070 |
| Process Example 9 | 0.060 |

### Example 2

Production of Porphyran Solution

Dried marine algae of Porphyra yezoensis from Seto Inland Sea was used as Porphyran material. Extraction was performed at 95° C. for 3 h with 165 kg of the dried marine algae in 1 t of boiling purified water. Then, after extraction residue was filtered and separated through a 30 mesh shifter, diatom earth and activated charcoal equivalent to 1% and 5% of the extract respectively were added to the extract, filter press was performed, and extraction residue, substances having staining properties and odor were completely removed. Next, the extract was passed through a resin tower of 1 m² filled with a strongly acidic cation-exchange resin DIAION SK-104 replaced with H+ type. Subsequently, the solution passed through the resin was adjusted to pH 6.5 by using arginine and subjected to vacuum concentration. The Porohyran solution of Bx. 5 was thus prepared.

### EXAMPLE 3

Production of Porphyran Added Body Soap

Based on respective compositions shown in Table 3, liquid body soaps were prepared. A panel consisting of 24 experimental subjects suffering from a topic dermatitis was instructed to use the each types of obtained body soap (about 6 g per use) every day as body soap, and evaluated their conditions of skin after one month's use. Every type of liquid body soap was tested by 8 panelists. Evaluation was carried out on scale of one to five referring to following items;
(1) moisturized condition of skin: 1. dry 2. slightly dry 3. normal 4. slightly moisturized 5. moisturized, and
(2) intensity of itching of skin: 1. itch increased, 2. no change 3. itch decreased somewhat 4. itch decreased 5. feel no itch. The mean values of marks evaluated by 24 panelists are shown in Table 5.

As is also apparent from Table 4, more excellent skin condition-improving effect was suggested by the product by formulation example 2 compounded with the Porphyran of the present invention than those by formulation examples 1 and 3 compounded with no Porphyran and conventional Porphyran, respectively.

TABLE 3

| | Compounding quantity (weight %) | | |
|---|---|---|---|
| | Formulation | Formulation | Formulation |
| Component | Ex. 1 | Ex. 2 | Ex. 3 |
| Porphyran solution of Ex. 2 | 0 | 2 | 0 |
| Porphyran solution of Ex. | 0 | 0 | 2 |
| 1-Process ex. 2 | | | |
| Lauric acid | 2.3 | 2.3 | 2.3 |
| Mysristic acid | 3.2 | 3.2 | 3.2 |
| 96% calucium hydrate | 1.5 | 1.5 | 1.5 |
| Sodium methyl cocoyl | 2.9 | 2.9 | 2.9 |
| taurate | | | |
| Palm oil fatty acid amide | 7.2 | 7.2 | 7.2 |
| propyl betaine | | | |
| Palm oil fatty acid | 3 | 3 | 3 |
| diethanol amid | | | |
| Edetate disodium | 0.2 | 0.2 | 0.2 |
| Parahydroxybensoate | 0.2 | 0.2 | 0.2 |
| methyl | | | |
| Salt | 1 | 1 | 1 |
| Distilled water | 78.5 | 76.5 | 76.5 |
| Total | 100 | 100 | 100 |

TABLE 4
Organoleptic Test of Body Soap

| | Moisturized | |
|---|---|---|
| | Condition of | Intensity of |
| | Skin | Itch of Skin |
| Formulation ex. 1 | 2.8 | 2.8 |
| Formulation ex. 2 | 4.2 | 4.4 |
| Formulation ex. 3 | 3.4 | 3.6 |

### EXAMPLE 4

Production of Porphyran Added Skin Lotion

Skin lotion was prepared according to the types of formulation shown in Table 5.

TABLE 5

| | Compounding quantity (weight %) | | |
|---|---|---|---|
| | Formulation | Formulation | Formulation |
| Component | Ex. 1 | Ex. 2 | Ex. 3 |
| Glycerin | 6.00 | 6.00 | 6.00 |
| 1.3-butylene glycol | 2.00 | 2.00 | 2.00 |
| Porphyran prepared in | 5.00 | 0.00 | 0.00 |
| Ex. 2 | | | |
| Porphyran of Ex. 1-process | 0.00 | 5.00 | 0.00 |
| ex. 2 | | | |
| Ethanol | 6.00 | 6.00 | 6.00 |
| PEG6000 | 0.50 | 0.50 | 0.50 |
| Polyoxyethylene methyl | 0.50 | 0.50 | 0.50 |
| glucoside | | | |
| Parahydroxybensoate ester | 0.15 | 0.15 | 0.15 |
| Polyoxyethylene hardned | 0.10 | 0.10 | 0.10 |
| caster oil | | | |
| Purified water | 79.75 | 79.75 | 84.75 |
| Total | 100.00 | 100.00 | 100.00 |

Each type of obtained skin lotion was bottled by 100 ml, whose preservation test was performed by being left under condition of RH 75% at 40° C. for three months. As a result, occurrence of precipitate was observed at the bottom of the container one month later in the lotion according to formulation example 2 compounded conventional Porphyran, which suggested that it was unsuitable for skin lotion, while other types of skin lotion according to formulation examples 1 and 3 were stable for three months. Next, organoleptic test was performed for types of skin lotion according to formulation examples 1 and 3 by 10 specialized panelists. Evaluation was carried out on scale of one to five referring to following items;
(1) moisturized condition of skin: 1. dry, 2. slightly dry, 3. normal, 4. slightly moisturized, 5. moisturized,
(2) skin smoothness: 1. rough, 2. slightly rough, 3. normal, 4. slightly smooth, 5. smooth, and
(3) skin tackiness: 1. tacky, 2. slightly tacky, 3. normal, 4. slightly refreshed, 5. refreshed.

The mean values of marks evaluated by 10 panelists are shown in Table 6.

As is apparent from Table 3, the product by formulation example 1 compounded Porphyran of the present invention showed more excellent activity of retaining moisture and smoothing skin than that by formulation example 3 compounded no Porphyran.

TABLE 6
Organoleptic Test of Skin Lotion

| | Moisturized | | |
|---|---|---|---|
| | condition of | Skin | Skin |
| | Skin | Smoothness | Tackiness |
| Formulation | 4.2 | 4.4 | 3.9 |
| ex. 1 | | | |
| Formulation | 1.9 | 2.8 | 3.7 |
| ex. 3 | | | |

### EXAMPLE 5

Production of Soft Drink Containing Porphyran

Soft drink containing Porphyran was produced by adding 150 ml of natural water, 20 ml of liquid sugar syrup, 5 ml of condensed grape fruit juice, and 0.1 ml of flavor to 50 ml of Porphyran solution of Example 1—process example 4.

### INDUSTRIAL APPLICABILITY

The present invention provides a method for producing Porphyran with modified bioactivity and physical property by a convenient method. The method allows not only providing modified Porphyran readily but also providing its practically useful application by using the modified Porphyran. As bioactivities and physical properties of Porphyran depend largely on the types of the salt of the sulphate group, setting the given salt as the salt of the sulphate makes it possible to provide stable Porphyran having notable inhibitory activity against hyaluronidase and the like, according to the present invention. The modified Porphyran of the present invention can be used as cosmetics and cleanser for hair or body having functions such as inhibitory activity hyaluronidase, by adding it to cosmetics and cleanser for hair or body. In addition Porphyran is a principal ingredient of Pophyra originally applied for food; hence practical use of Porphyran can be achieved as food with health-promoting benefit safely produced by adding it to drink or food.

1. A method for producing modified Porphyran, the method of comprising the steps of:
dissociating a salt from a sulphate group in Porphyran; and removing said salt.

2. A method for producing modified Porphyran, the method comprising the steps of:
dissociating a salt from a sulphate group in Porphyran, removing said salt and then altering the sulphate group, with the use of a given salt, into a sulphate salt of the given salt; or
converting a salt of a sulphate group in Porphyran into a sulphate salt of a given salt by ion exchange.

3. The method for producing modified Porphyran according to claim 1 or 2, wherein salt dissociation from the sulphate group in Porphyran and removal of the salt, or conversion of the salt of sulphate group in Porphyran by ionic exchange is performed with a cation-exchange resin.

4. The method for producing modified Porphyran according to claim 2, wherein the sulphate salt of the given salt is sodium salt, potassium salt, calcium salt, arginine salt, omithine salt, or histidine salt of sulphuric acid.

5. A modified Porphyran wherein the Porphyran is produced by the method for producing according to claim 1, thereby making the Porphyran having a free sulphate group wherein a salt thereof is dissociated.

6. A modified Porphyran, wherein it is produced by the method for producing according to claim 2, thereby the salt of the sulphate group in Porphyran is converted to the given salt.

7. The modified Porphyran according to claim 6, wherein the given salt is sodium salt, and shows high stability and enhanced adsorption to skin when it is brought into solution.

8. The modified Porphyran according to claim 6, wherein the given salt is arginine salt, and shows high stability and enhanced adsorption to skin when it is brought into solution.

9. A hyaluronidase activity-inhibitory agent wherein its active ingredient is the modified Porphyran according to claim 5 or 6.

10. A cholesterol-lowering agent wherein its active ingredient is the modified Porphyran according to claim 5 or 6.

11. An immunostimulant wherein its active ingredient is the modified Porphyran according to claim 5 or 6.

12. A cosmetic or a cleanser for hair or body, to which the modified Porphyran according to claim 5 or 6 is added.

13. A food or a drink, to which modified Porphyran according to claim 5 or 6 is added.
